(19) United States
(12) Patent Application Publication
(10) Pub. No.: US 2026/0053711 A1
(43) Pub. Date: Feb. 26, 2026

(54) SEMI-SOLID PHARMACEUTICAL COMPOSITION MICRO-DISPENSER

(71) Applicant: AZURA OPHTHALMICS LTD., Tel Aviv (IL)

(72) Inventors: Yair ALSTER, Tel Aviv (IL); Omer RAFAELI, Tel Aviv (IL); Hila EPSTEIN-BARASH, Tel Aviv (IL); Charles BOSWORTH, Las Vegas, NV (US); Marc GLEESON, Longueville (AU)

(21) Appl. No.: 19/304,525

(22) Filed: Aug. 19, 2025

Related U.S. Application Data
(60) Provisional application No. 63/685,156, filed on Aug. 20, 2024.

(51) Int. Cl. A61J 7/00; A61K 9/00; A61K 9/06; A61K 33/04; A61P 27/02

(52) U.S. Cl. CPC A61J 7/0076; A61K 9/0048; A61K 9/06; A61K 33/04; A61P 27/02

(57) ABSTRACT

Provided in certain embodiments herein are systems and devices, suitable for precisely dispensing small amounts of a semi-solid composition multiple times.

### CROSS-REFERENCE

This application claims the benefit of U.S. Provisional Application No. 63/685,156, filed Aug. 20, 2024, which application is incorporated herein by reference in its entirety.

### BACKGROUND OF THE INVENTION

A variety of semi-solid pharmaceutical or cosmetic compositions are currently being sold and tested for the treatment of a multitude of medical conditions or for cosmetic reasons. Generally, such compositions involve use of imprecise volumes for topical administration. In some instances, liquid compositions can be dispensed in precise amounts for topical administration, such as through the use of dispensing devices, such as eye droppers. Precise volumes for dispensing semi-solid compositions, however, are less common, especially when dispensing very small amounts of semi-solid compositions.

### SUMMARY OF THE INVENTION

Provided in certain embodiments herein are systems and devices, suitable for precisely dispensing small amounts of a semi-solid composition multiple times.

In some embodiments, provided herein is a system for dispensing a (e.g., semi-solid) pharmaceutical composition. In some embodiments, the system comprises a (e.g., semi-solid) pharmaceutical composition and a dispenser. In specific embodiments, the dispenser comprises a chamber configured to contain a first volume (V₁) of a (e.g., semi-solid) pharmaceutical composition, an outlet in fluid communication with the chamber, and a device (e.g., a plunger) configured to displace a portion of a volume of V₁ in the chamber. In certain embodiments, when the device (e.g., plunger) displaces a portion of a volume of V₁ in the chamber, a portion of the (e.g., semi-solid) pharmaceutical composition is dispensed from the outlet of the device. In some embodiments, the volume of the (e.g., semi-solid) pharmaceutical composition dispensed from the outlet (V₂) is the same volume displaced from the chamber. In some embodiments, the pharmaceutical composition is fluid, liquid, or semi-solid. In specific embodiments, the pharmaceutical composition is semi-solid.

In some embodiments, provided herein is a system for dispensing a (e.g., semi-solid) pharmaceutical composition. In some embodiments, the system comprises a (e.g., semi-solid) pharmaceutical composition and a dispenser. In specific embodiments, the dispenser comprises a chamber configured to contain a first volume (V₁) of a (e.g., semi-solid) pharmaceutical composition, an outlet in fluid communication with the chamber, and a device (e.g., a plunger) configured to dispense a precise volume of the (e.g., semi-solid) pharmaceutical composition from the outlet. In more specific embodiments, the dispenser comprises a chamber configured to contain a first volume (V₁) of a (e.g., semi-solid) pharmaceutical composition, an outlet in fluid communication with the chamber, and a device (e.g., a plunger) configured to expel the semi-solid pharmaceutical composition from the chamber and dispense a precise volume of the (e.g., semi-solid) pharmaceutical composition from the outlet.

Provided in certain embodiments herein is a system for dispensing a semi-solid pharmaceutical composition, the system comprising:
a. the semi-solid pharmaceutical composition; and
b. a dispenser comprising (i) a chamber configured to contain a first volume (V₁) of the semi-solid pharmaceutical composition, (ii) an outlet in fluid communication with the chamber, and (iii) a device (e.g., a plunger) configured to expel a portion of the semi-solid pharmaceutical composition from the chamber and dispense a precise volume of the semi-solid pharmaceutical composition from the outlet,
wherein the portion of the semi-solid pharmaceutical composition expelled from the chamber has a second volume (V₂), the device is configured to expel a maximum number (Nₘ) of second volume portions of the semi-solid pharmaceutical composition from the chamber, and V₁>V₂×Nₘ.

In some embodiments, the semi-solid pharmaceutical composition is configured within the chamber.

In some embodiments, Nₘ is about 120 or less (e.g., 10-120). In some embodiments, Nₘ is about 100 or less (e.g., 15-90). In some embodiments, Nₘ is about 60 or less (e.g., 20-60 or 20-40). In some embodiments, Nₘ is about 30 or less (e.g., 1 months' worth with daily use).

In some embodiments, V₁ is at least 2 times V₂×Nₘ. In some embodiments, V₁ is at least 5 times V₂×Nₘ. In some embodiments, V₁ is at least 10 times V₂×Nₘ.

In some embodiments, V₁ is about 100 μL or less. In some embodiments, V₁ is about 50 μL or less. In some embodiments, V₁ is about 25 μL or less. In some embodiments, V₁ is about 5 μL. In some embodiments, V₁ is about 0.1 μL to about 100 μL. In some embodiments, V₁ is about 0.5 μL to about 50 μL. In some embodiments, V₁ is about 1 μL to about 25 μL.

In some embodiments, the semi-solid pharmaceutical composition is an ointment, a gel, a cream, or a paste. In some embodiments, the semi-solid pharmaceutical composition is an ointment.

In some embodiments, the semi-solid pharmaceutical composition comprises a keratolytic agent. In some embodiments, the semi-solid pharmaceutical composition comprises a keratolytic agent in an amount, wherein V₂ of the semi-solid pharmaceutical composition comprises a therapeutically effective amount of the keratolytic agent. In some embodiments, the semi-solid pharmaceutical composition comprises a keratolytic agent in an amount greater than or equal to about 0.1% by weight (wt. %). In some embodiments, the semi-solid pharmaceutical composition comprises a keratolytic agent in an amount of about 0.1 wt. % to about 10 wt. %. In some embodiments, the semi-solid pharmaceutical composition comprises a keratolytic agent in an amount of about 0.1 wt. % to about 2 wt. %. In some embodiments, the semi-solid pharmaceutical composition comprises a keratolytic agent in an amount less than or equal to about 1 wt. %. In some embodiments, the semi-solid pharmaceutical composition comprises a keratolytic agent in an amount of about 1 wt. %. In some embodiments, the semi-solid pharmaceutical composition comprises a keratolytic agent in an amount of about 0.5 wt. %.

In some embodiments, the semi-solid pharmaceutical composition comprises selenium disulfide (SeS2). In some embodiments, the semi-solid pharmaceutical composition comprises selenium disulfide (SeS2) in an amount, wherein V₂ of the semi-solid pharmaceutical composition comprises a therapeutically effective amount of the selenium disulfide (SeS2).

In some embodiments, the semi-solid pharmaceutical composition comprises SeS2 in an amount greater than or equal to about 0.1% by weight (wt. %). In some embodiments, the semi-solid pharmaceutical composition comprises SeS2 in an amount of about 0.1 wt. % to about 10 wt. %. In some embodiments, the semi-solid pharmaceutical composition comprises SeS2 in an amount of about 0.1 wt. % to about 2 wt. %. In some embodiments, the semi-solid pharmaceutical composition comprises SeS2 in an amount less than or equal to about 1 wt. %. In some embodiments, the semi-solid pharmaceutical composition comprises selenium disulfide in an amount of about 0.5 wt. % to about 1 wt. %. In some embodiments, the semi-solid pharmaceutical composition comprises SeS2 in an amount of about 1 wt. %. In some embodiments, the semi-solid pharmaceutical composition comprises SeS2 in an amount of about 0.5 wt. %.

In some embodiments, the keratolytic agent is selected from the group consisting of benzoyl peroxide, coal tar, dithranol, salicylic acid, selenium disulfide, alpha-hydroxy acid, urea, lactic acid, boric acid, retinoic acid, sodium thioglycolate, allantoin, zinc pyrithione, zinc L-pyrrolidone carboxylate, seleocysteine, selenomethionine, captopril, zofenopril, tiopronin, penicillamine, L-cysteine, N-acetyl cysteine (NAC), gluthatione, dithiothreitol, thiorphan, cysteamine, bucillamine, dimercaprol, 1,1-ethanedithiol, dimercaptosuccinic acid, furan-2-ylmethanethiol, omapatrilat, ovothiol A, rentiapril, thiosalicylic acid, tixocortol, mycothiol, coenzyme A, coenzyme B, disulfiram, psammaplin A, dixanthogen, pantethine, fursultiamine, octotiamine, sulbutiamine, prosultiamine, thiram, lipoic acid, lenthionine, ajoene, allicin, gemopatrilat, thioethanol, thiophospholipid, thiocholesterol, 12-mercaptododecanoic acid, 23-(9-mercaptononyl)-3,6,9,12,15,18,21-heptaoxatricosanoic acid, and sulfanegen. In some embodiments, the keratolytic agent is selected from the group consisting of benzoyl peroxide, coal tar, dithranol, salicylic acid, selenium disulfide, N-acetyl cysteine (NAC), bucillamine, captopril, alpha-hydroxy acid, urea, lactic acid, sodium thioglycolate, zinc pyrithione, and zinc L-pyrrolidone carboxylate.

In some embodiments, the system and/or the semi-solid pharmaceutical composition is used for treating a disease or disorder in or around an eye. In some embodiments, the system and/or the semi-solid pharmaceutical composition is used for treating a disease or disorder in or around an eye, wherein the disease or disorder in or around an eye is meibomian gland dysfunction (MGD), blepharitis, seborrheic blepharitis, Demodex infestation, dry eye disease (DED), hyperkeratosis, dermatitis, keratitis, contact lens discomfort, lid wiper epitheliopathy (LWE), Keratoconjunctivitis Sicca, Sjogren's Syndrome, or ocular rosacea. In some embodiments, the disease or disorder in or around an eye is meibomian gland dysfunction (MGD) or dry eye disease (DED).

Provided in some embodiments herein is a method of treating meibomian gland dysfunction (MGD) or dry eye disease (DED), the method comprising dispensing a precise volume of the semi-solid pharmaceutical composition from any system described herein and administering the dispensed semi-solid pharmaceutical composition in or around an eye of the individual.

Provided in some embodiments herein is a system for dispensing a semi-solid pharmaceutical composition, the system comprising:
a. the semi-solid pharmaceutical composition comprising a therapeutically effective concentration of selenium disulfide (SeS2); and
b. a dispenser configured to dispense precise volume of the pharmaceutical composition, the volume being sufficient to treat a disease or disorder in or around the eye of an individual when the volume is administered in or around the eye of an individual, wherein the dispenser comprises a barrel chamber having an outlet and a plunger configured to dispense the pharmaceutical composition from the chamber through the outlet (e.g., as described in US 2020/0147323, US 2020/0147322, US 2022/0387731, US 2022/0134021, WO 2020/102444, and WO2020/102446, all of which are incorporated herein by reference.)

Provided in some embodiments herein is a method of treating a disease or disorder in or around an eye of an individual, the method comprising dispensing a precise volume of the semi-solid pharmaceutical composition from any system provided herein and administering the dispensed semi-solid pharmaceutical composition in or around the eye of the individual.

Provided in some embodiments herein is a method of treating a disease or disorder in or around an eye of an individual, the method comprising dispensing a precise volume of the semi-solid pharmaceutical composition from any system provided herein and administering the dispensed semi-solid pharmaceutical composition in or around the eye of the individual for up to Nₘ times.

Provided in some embodiments herein is a method of treating a disease or disorder in or around an eye of an individual, the method comprising dispensing a precise volume of the semi-solid pharmaceutical composition from any system provided herein and administering the dispensed semi-solid pharmaceutical composition in or around the eye of the individual for a total of 2 to Nₘ times.

Provided in certain embodiments herein is a method for dispensing a semi-solid pharmaceutical composition. In some embodiments, the method comprises providing a system (e.g., any system provided herein), such as a system comprising the semi-solid pharmaceutical composition and a dispenser. In some embodiments, the method further comprises dispensing a precise volume of the semi-solid pharmaceutical composition from the dispenser. In some embodiments, the dispenser comprises (i) a chamber configured to contain a first volume (V₁) of the semi-solid pharmaceutical composition, and (ii) an outlet in fluid communication with the chamber. In some embodiments, the dispenser is configured to expel a portion of the semi-solid pharmaceutical composition from the chamber and dispense the precise volume of the semi-solid pharmaceutical composition from the outlet. In some embodiments, the precise volume has a second volume (V₂). In some embodiments, the dispenser is configured to expel a maximum number (Nₘ) of second volume portions of the semi-solid pharmaceutical composition from the chamber. In some embodiments, the method further comprises administering the at least a portion of the precise volume of the semi-solid pharmaceutical composition to an individual (e.g., according in a method of treating provided herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates an exemplary system for administering a semi-solid pharmaceutical composition.

FIG. 2A illustrates an exemplary plunger rack that can be used in an exemplary system for administering a semi-solid pharmaceutical composition. FIG. 2B illustrates an exemplary counter wheel that can be used in combination with a plunger rack in an exemplary system for administering a semi-solid pharmaceutical composition.

### DETAILED DESCRIPTION OF THE INVENTION

### Certain Definitions

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

The term “optional” or “optionally” denotes that a subsequently described event or circumstance can but need not occur, and that the description includes instances where the event or circumstance occurs and instances in which it does not.

In some embodiments, provided herein is a system for dispensing a (e.g., semi-solid) pharmaceutical composition. In some embodiments, the system comprises a (e.g., semi-solid) pharmaceutical composition and a dispenser. In specific embodiments, the dispenser comprises a chamber configured to contain a first volume (V₁) of a (e.g., semi-solid) pharmaceutical composition, an outlet in fluid communication with the chamber, and a device (e.g., a plunger) configured to displace a portion of a volume of V₁ in the chamber. In certain embodiments, when the device (e.g., plunger) displaces a portion of a volume of V₁ in the chamber, a portion of the (e.g., semi-solid) pharmaceutical composition is dispensed from the outlet of the device. In some embodiments, the volume of the (e.g., semi-solid) pharmaceutical composition dispensed from the outlet (V₂) is the same volume displaced from the chamber. In some embodiments, the pharmaceutical composition is fluid, liquid, or semi-solid. In specific embodiments, the pharmaceutical composition is semi-solid.

In some embodiments, provided herein is a system for dispensing a (e.g., semi-solid) pharmaceutical composition. In some embodiments, the system comprises a (e.g., semi-solid) pharmaceutical composition and a dispenser. In specific embodiments, the dispenser comprises a chamber configured to contain a first volume (V₁) of a (e.g., semi-solid) pharmaceutical composition, an outlet in fluid communication with the chamber, and a device (e.g., a plunger) configured to dispense a precise volume of the (e.g., semi-solid) pharmaceutical composition from the outlet. In more specific embodiments, the dispenser comprises a chamber configured to contain a first volume (V₁) of a (e.g., semi-solid) pharmaceutical composition, an outlet in fluid communication with the chamber, and a device (e.g., a plunger) configured to expel the semi-solid pharmaceutical composition from the chamber and dispense a precise volume of the (e.g., semi-solid) pharmaceutical composition from the outlet.

In some embodiments, a volume of (e.g., semi-solid) pharmaceutical composition expelled from the chamber and/or dispensed from the outlet has a second volume (V₂). In specific embodiments, the volume of (e.g., semi-solid) pharmaceutical composition expelled from the chamber and dispensed from the outlet are the same and have a second volume (V₂).

In certain embodiments, a device provided herein is configured to expel a maximum number (Nₘ) of second volume portions of (e.g., semi-solid) pharmaceutical composition from the chamber and/or dispense a maximum number (Nₘ) of second volume portions of (e.g., semi-solid) pharmaceutical composition from the outlet. In specific embodiments, a device provided herein is configured to expel a maximum number (Nₘ) of second volume portions of (e.g., semi-solid) pharmaceutical composition from the chamber. In some specific embodiments, a device provided herein is configured to dispense a maximum number (Nₘ) of second volume portions of (e.g., semi-solid) pharmaceutical composition from the outlet. Generally, the maximum number (Nₘ) of second volume (V₂) portions expelled from the chamber and the maximum number (Nₘ) of second volume (V₂) portions dispensed in the outlet are the same.

In some embodiments, a device provided herein is configured to expel a maximum number (Nₘ) of second volume portions of (e.g., semi-solid) pharmaceutical composition from the chamber. In some embodiments, the maximum number (Nₘ) of second volume portions expelled from the chamber is any suitable or desirable number. In some embodiments, the maximum number (Nₘ) of second volume portions expelled from the chamber is about 120 or less. In some embodiments, the maximum number (Nₘ) of second volume portions expelled from the chamber is about 100 or less. In some embodiments, the maximum number (Nₘ) of second volume portions expelled from the chamber is about 90 or less. In some embodiments, the maximum number (Nₘ) of second volume portions expelled from the chamber is about 60 or less. In some embodiments, the maximum number (Nₘ) of second volume portions expelled from the chamber is about 45 or less. In some embodiments, the maximum number (Nₘ) of second volume portions expelled from the chamber is about 30 or less. In some embodiments, the maximum number (Nₘ) of second volume portions expelled from the chamber is about 15 or less. In some embodiments, the maximum number (Nₘ) of second volume portions expelled from the chamber is about 7 or less. In some embodiments, the maximum number (Nₘ) of second volume portions expelled from the chamber is about 7 to about 120. In some embodiments, the maximum number (Nₘ) of second volume portions expelled from the chamber is about 7 to about 100. In some embodiments, the maximum number (Nₘ) of second volume portions expelled from the chamber is about 10 to about 90. In some embodiments, the maximum number (Nₘ) of second volume portions expelled from the chamber is about 15 to about 60. In some embodiments, the maximum number (Nₘ) of second volume portions expelled from the chamber is about 15 to about 45. In some embodiments, the maximum number (Nₘ) of second volume portions expelled from the chamber is about 15 to about 30. In some embodiments, the maximum number (Nₘ) of second volume portions expelled from the chamber is about 30. In some embodiments, the maximum number (Nₘ) of second volume portions expelled from the chamber is about 15. In some embodiments, the maximum number (Nₘ) of second volume portions expelled from the chamber is about 7. In certain embodiments, a limited maximum number of second volume portions expelled from a device provided herein is desirable in order to reduce chances of product degradation over time, reduce chances of product contamination, etc.

In some embodiments, a device provided herein is configured to dispense a maximum number (Nₘ) of second volume portions of (e.g., semi-solid) pharmaceutical composition from the outlet. In some embodiments, the maximum number (Nₘ) of second volume portions dispensed from the outlet is any suitable or desirable number. In some embodiments, the maximum number (Nₘ) of second volume portions dispensed from the outlet is about 120 or less. In some embodiments, the maximum number (Nₘ) of second volume portions dispensed from the outlet is about 100 or less. In some embodiments, the maximum number (Nₘ) of second volume portions dispensed from the outlet is about 90 or less. In some embodiments, the maximum number (Nₘ) of second volume portions dispensed from the outlet is about 60 or less. In some embodiments, the maximum number (Nₘ) of second volume portions dispensed from the outlet is about 45 or less. In some embodiments, the maximum number (Nₘ) of second volume portions dispensed from the outlet is about 30 or less. In some embodiments, the maximum number (Nₘ) of second volume portions dispensed from the outlet is about 15 or less. In some embodiments, the maximum number (Nₘ) of second volume portions dispensed from the outlet is about 7 or less. In some embodiments, the maximum number (Nₘ) of second volume portions dispensed from the outlet is about 7 to about 120. In some embodiments, the maximum number (Nₘ) of second volume portions dispensed from the outlet is about 7 to about 100. In some embodiments, the maximum number (Nₘ) of second volume portions dispensed from the outlet is about 10 to about 90. In some embodiments, the maximum number (Nₘ) of second volume portions dispensed from the outlet is about 15 to about 60. In some embodiments, the maximum number (Nₘ) of second volume portions dispensed from the outlet is about 15 to about 45. In some embodiments, the maximum number (Nₘ) of second volume portions dispensed from the outlet is about 15 to about 30. In some embodiments, the maximum number (Nₘ) of second volume portions dispensed from the outlet is about 30. In some embodiments, the maximum number (Nₘ) of second volume portions dispensed from the outlet is about 15. In some embodiments, the maximum number (Nₘ) of second volume portions dispensed from the outlet is about 7. In certain embodiments, a limited maximum number of second volume portions dispensed from a device provided herein is desirable in order to reduce chances of product degradation over time, reduce chances of product contamination, etc.

In some embodiments, the volume of (e.g., semi-solid) pharmaceutical composition expelled from the chamber is any suitable volume. In certain embodiments, the volume of (e.g., semi-solid) pharmaceutical composition expelled from the chamber is about 100 μL or less. In certain embodiments, the volume of (e.g., semi-solid) pharmaceutical composition expelled from the chamber is about 80 μL or less. In certain embodiments, the volume of (e.g., semi-solid) pharmaceutical composition expelled from the chamber is about 60 μL or less. In certain embodiments, the volume of (e.g., semi-solid) pharmaceutical composition expelled from the chamber is about 50 μL or less. In certain embodiments, the volume of (e.g., semi-solid) pharmaceutical composition expelled from the chamber is about 25 μL or less. In certain embodiments, the volume of (e.g., semi-solid) pharmaceutical composition expelled from the chamber is about 10 μL or less. In certain embodiments, the volume of (e.g., semi-solid) pharmaceutical composition expelled from the chamber is about 5 μL or less. In certain embodiments, the volume of (e.g., semi-solid) pharmaceutical composition expelled from the chamber is about 0.1 μL to about 100 μL. In certain embodiments, the volume of (e.g., semi-solid) pharmaceutical composition expelled from the chamber is about 0.5 μL to about 100 μL. In certain embodiments, the volume of (e.g., semi-solid) pharmaceutical composition expelled from the chamber is about 1 μL to about 80 μL. In certain embodiments, the volume of (e.g., semi-solid) pharmaceutical composition expelled from the chamber is about 1 μL to about 50 μL. In certain embodiments, the volume of (e.g., semi-solid) pharmaceutical composition expelled from the chamber is about 1 μL to about 25 μL. In certain embodiments, the volume of (e.g., semi-solid) pharmaceutical composition expelled from the chamber is about 2 μL to about 25 μL. In certain embodiments, the volume of (e.g., semi-solid) pharmaceutical composition expelled from the chamber is about 2 μL to about 10 μL. In certain embodiments, the volume of (e.g., semi-solid) pharmaceutical composition expelled from the chamber is about 5 μL. In certain embodiments, the volume of (e.g., semi-solid) pharmaceutical composition expelled from the chamber is about 2 μL. In certain embodiments, the volume of (e.g., semi-solid) pharmaceutical composition expelled from the chamber is about 1 μL.

In some embodiments, the volume of (e.g., semi-solid) pharmaceutical composition dispensed from the outlet is any suitable volume. In certain embodiments, the volume of (e.g., semi-solid) pharmaceutical composition dispensed from the outlet is about 100 μL or less. In certain embodiments, the volume of (e.g., semi-solid) pharmaceutical composition dispensed from the outlet is about 80 μL or less. In certain embodiments, the volume of (e.g., semi-solid) pharmaceutical composition dispensed from the outlet is about 60 μL or less. In certain embodiments, the volume of (e.g., semi-solid) pharmaceutical composition dispensed from the outlet is about 50 μL or less. In certain embodiments, the volume of (e.g., semi-solid) pharmaceutical composition dispensed from the outlet is about 25 μL or less. In certain embodiments, the volume of (e.g., semi-solid) pharmaceutical composition dispensed from the outlet is about 10 μL or less. In certain embodiments, the volume of (e.g., semi-solid) pharmaceutical composition dispensed from the outlet is about 5 μL or less. In certain embodiments, the volume of (e.g., semi-solid) pharmaceutical composition dispensed from the outlet is about 0.1 μL to about 100 μL. In certain embodiments, the volume of (e.g., semi-solid) pharmaceutical composition dispensed from the outlet is about 0.5 μL to about 100 μL. In certain embodiments, the volume of (e.g., semi-solid) pharmaceutical composition dispensed from the outlet is about 1 μL to about 80 μL. In certain embodiments, the volume of (e.g., semi-solid) pharmaceutical composition dispensed from the outlet is about 1 μL to about 50 μL. In certain embodiments, the volume of (e.g., semi-solid) pharmaceutical composition dispensed from the outlet is about 1 μL to about 25 μL. In certain embodiments, the volume of (e.g., semi-solid) pharmaceutical composition dispensed from the outlet is about 2 μL to about 25 μL. In certain embodiments, the volume of (e.g., semi-solid) pharmaceutical composition dispensed from the outlet is about 2 μL to about 10 μL. In certain embodiments, the volume of (e.g., semi-solid) pharmaceutical composition dispensed from the outlet is about 5 μL. In certain embodiments, the volume of (e.g., semi-solid) pharmaceutical composition dispensed from the outlet is about 2 μL. In certain embodiments, the volume of (e.g., semi-solid) pharmaceutical composition dispensed from the outlet is about 1 μL.

In some embodiments, a dispenser provided herein comprises a chamber having any suitable volume. In certain embodiments, the chamber has a volume of about 0.1 mL to about 100 mL. In certain embodiments, the chamber has a volume of about 0.2 mL to about 50 mL. In certain embodiments, the chamber has a volume of about 0.5 mL to about 25 mL. In certain embodiments, the chamber has a volume of about 1 mL to about 10 mL.

In some embodiments, a system provided herein comprises a dispenser comprising a chamber configured to contain any suitable volume of a (e.g., semi-solid) pharmaceutical composition. In specific embodiments, the (e.g., semi-solid) pharmaceutical composition is configured within the chamber. In certain embodiments, the chamber is configured to contain a volume of about 0.1 mL to about 100 mL of a (e.g., semi-solid) pharmaceutical composition. In certain embodiments, the chamber is configured to contain a volume of about 0.2 mL to about 50 mL of a (e.g., semi-solid) pharmaceutical composition. In certain embodiments, the chamber is configured to contain a volume of about 1 mL to about 10 mL of a (e.g., semi-solid) pharmaceutical composition. In some instances, a dispenser having a chamber containing a larger volume of a (e.g., semi-solid) pharmaceutical composition facilitates manufacturing a system comprising the dispenser and the (e.g., semi-solid) pharmaceutical composition, precision dispensing of the (e.g., semi-solid) pharmaceutical composition, etc.

In some embodiments, a system comprises a dispenser comprising a chamber configured to contain a first volume (V₁) of a (e.g., semi-solid) pharmaceutical composition. In some embodiments, a dispenser comprises a chamber configured to contain a first volume (V₁) of a (e.g., semi-solid) pharmaceutical composition. In some embodiments, a dispenser comprises a chamber having a first volume (V₁).

In some embodiments, a portion of a (e.g., semi-solid) pharmaceutical composition expelled from a chamber of a system or dispenser provided herein has a second volume (V₂). In some embodiments, a (e.g., semi-solid) pharmaceutical composition dispensed from an outlet of a system or dispenser provided herein has a second volume (V₂).

In some embodiments, a device is configured to expel a maximum number (Nₘ) of second volume portions of the semi-solid pharmaceutical composition from a chamber of a dispenser provided herein. In some embodiments, a dispenser provided herein is configured to dispense a maximum number (Nₘ) of second volume portions of a (e.g., semi-solid) pharmaceutical composition from a system provided herein.

In some embodiments, V₁>V₂×Nₘ. In certain embodiment, V₁ is at least 1.1 times V₁×Nₘ. In certain embodiment, V₁ is at least 1.2 times V₂×Nₘ. In certain embodiment, V₁ is at least 1.3 times V₁×Nₘ. In certain embodiment, V₁ is at least 1.5 times V₁×Nₘ. In certain embodiment, V₁ is at least 2 times V₂×Nₘ. In some embodiments, V₁ is at least 5 times V₁×Nₘ. In some embodiments, V₁ is at least 10 times V₁×Nₘ. In certain embodiment, V₁ is 1.1 to 100 times V₂×Nₘ. In certain embodiment, V₁ is 1.5 to 100 times V₂×Nₘ. In certain embodiment, V₁ is 2 to 100 times V₂×Nₘ. In certain embodiment, V₁ is 5 to 50 times V₂×Nₘ. In certain instances, an excess of V₁ relative to V₂×Nₘ facilitates precise dispensing of volume of (e.g., semi-solid) pharmaceutical composition from the outlet of the device over the full usage life of the device (the maximum number of dispensed volumes), even when the dispensed volumes are very small, such as less than 100 μL.

In some embodiments, a system or dispenser provided herein is configured to dispense a precise volume of a (e.g., semi-solid) pharmaceutical composition a number of times. In some embodiments, a system or dispenser provided herein is configured to repeatedly dispense a precise volume of a (e.g., semi-solid) pharmaceutical composition. In specific embodiments, a system or dispenser repeatedly dispenses a precise volume when each dispensed volume from the system or dispenser is within 50% of a selected (or average) volume. In specific embodiments, a system or dispenser repeatedly dispenses a precise volume when each dispensed volume from the system or dispenser is within 25% of a selected (or average) volume. In specific embodiments, a system or dispenser repeatedly dispenses a precise volume when each dispensed volume from the system or dispenser is within 20% of a selected (or average) volume. In specific embodiments, a system or dispenser repeatedly dispenses a precise volume when each dispensed volume from the system or dispenser is within 10% of a selected (or average) volume. In specific embodiments, a system or dispenser repeatedly dispenses a precise volume when each dispensed volume from the system or dispenser is within 5% of a selected (or average) volume.

In some embodiments, provided herein is a system for dispensing a (e.g., semi-solid) pharmaceutical composition. In some embodiments, the system comprises a (e.g., semi-solid) pharmaceutical composition and a dispenser. In specific embodiments, the dispenser comprises a chamber configured to contain a first volume (V₁) of a (e.g., semi-solid) pharmaceutical composition, an outlet in fluid communication with the chamber, and a device (e.g., a plunger) configured to displace a portion of a volume of V₁ in the chamber. In certain embodiments, when the device (e.g., plunger) displaces a portion of a volume of V₁ in the chamber, a portion of the (e.g., semi-solid) pharmaceutical composition is dispensed from the outlet of the device. In some embodiments, the volume of the (e.g., semi-solid) pharmaceutical composition dispensed from the outlet (V₂) is the same volume displaced from the chamber. In some embodiments, the pharmaceutical composition is fluid, liquid, or semi-solid. In specific embodiments, the pharmaceutical composition is semi-solid.

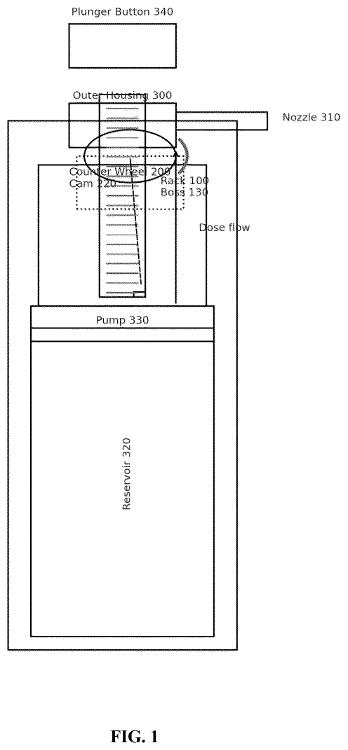

Any suitable device can be used in association with a system provided herein. FIG. 1 illustrates an exemplary device used in a system provided herein. In some instances, the device comprises a plunger-rack 100 comprises a rack body 110 carrying a predetermined number of rack teeth 120 (with twenty rack teeth being illustrated) and a stop boss 130. In some instances, the number of rack teeth corresponds with the number of total number of dispensed volumes capable of being dispensed by the device. In other instances, the number of rack teeth may correspond with a number larger than the total number of dispensed volumes capable of being dispensed by the device. In some instances, the rack translates axially within a housing 300 when a user depresses a plunger button 340.

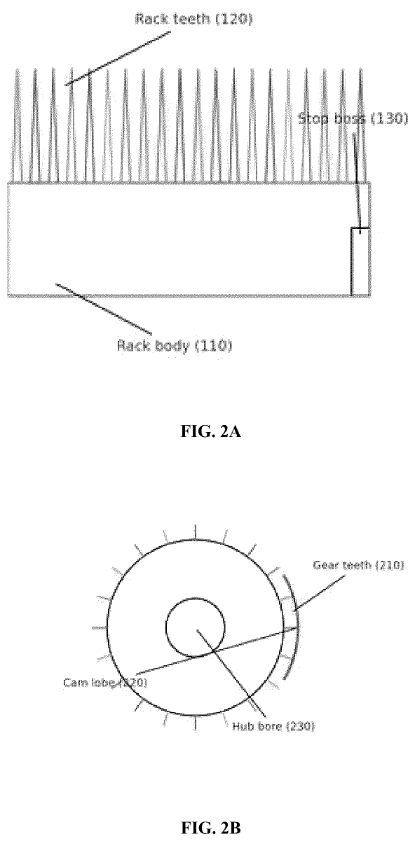

In some instances, a device provided herein comprises a counter wheel 200. In some instances, the counter wheel comprises a number of gear teeth 210. In some instances, the number of gear teeth 210 is equal to the number of rack teeth 120 of the plunger rack 100. In other instances, the number of gear teeth 210 may be greater or less than the number of rack teeth 120. FIG. 2A and FIG. 2B illustrate an exemplary embodiment of a plunger rack 100 and a counter wheel 200, with twenty rack teeth 120 and twenty gear teeth 210, respectively. In some instances, gear teeth 210 are journalled within the neck of the housing and meshes with rack teeth 120. In some instances, an integral cam lobe 220 protrudes axially from one face of the wheel.

In some instances, each press of the plunger button 340 strokes the rack 100 one time, which in turn indexes the wheel by one tooth (e.g., 18 degrees for a wheel 200 with twenty gear teeth 210). In some instances, after a total number of strokes of the wheel equaling the total number of gear teeth, the wheel completes one revolution. In some instances, the device is configured that upon the completion of one revolution, the cam lobe 220 is positioned directly adjacent to a stop boss 130. Once positioned adjacent to the stop boss, additional attempted strokes can cause the boss to abut the cam, thereby preventing further depression of the rack and concomitantly disabling pump head 330. In some instances, lock-out is mechanical and irreversible without destructive disassembly. In some instances, ointment is metered by pump head 330 and conveyed from a reservoir 320 through a stem to a nozzle 310 only when the rack is permitted to complete its stroke.

In some embodiments, a system provided herein comprises any suitable pharmaceutical composition. In specific embodiments, the pharmaceutical composition is a fluid, liquid, or semi-solid pharmaceutical composition. In more specific embodiments, the pharmaceutical composition is a semi-solid pharmaceutical composition. In still more specific embodiments, the pharmaceutical composition is an ointment, a gel, a cream, or a paste. In some embodiments, the pharmaceutical composition is an ointment.

In certain embodiments, a pharmaceutical composition provide herein comprises any suitable ingredients, including active pharmaceutical ingredient(s), carrier(s), excipient(s), etc. In some embodiments, a pharmaceutical composition provided herein comprises a keratolytic agent. In some embodiments, a pharmaceutical composition provided herein comprises any suitable concentration of an active pharmaceutical ingredient, such as a keratolytic agent. In some embodiments, a pharmaceutical composition provided herein comprises about 0.1 wt. % or more of an active pharmaceutical ingredient. In some embodiments, a pharmaceutical composition provided herein comprises about 0.2 wt. % or more of an active pharmaceutical ingredient. In some embodiments, a pharmaceutical composition provided herein comprises about 0.5 wt. % or more of an active pharmaceutical ingredient. In some embodiments, a pharmaceutical composition provided herein comprises about 1 wt. % or more of an active pharmaceutical ingredient. In some embodiments, a pharmaceutical composition provided herein comprises about 0.1 wt. % to about 10 wt. % of an active pharmaceutical ingredient. In some embodiments, a pharmaceutical composition provided herein comprises about 0.1 wt. % to about 5 wt. % of an active pharmaceutical ingredient. In some embodiments, a pharmaceutical composition provided herein comprises about 0.1 wt. % to about 2 wt. % of an active pharmaceutical ingredient. In some embodiments, a pharmaceutical composition provided herein comprises about 0.5 wt. % to about 5 wt. % of an active pharmaceutical ingredient. In some embodiments, a pharmaceutical composition provided herein comprises about 0.5 wt. % of an active pharmaceutical ingredient. In some embodiments, a pharmaceutical composition provided herein comprises about 1 wt. % of an active pharmaceutical ingredient.

In some embodiments, a system or dispenser provided herein is configured to dispense a volume of a pharmaceutical composition, such as provided herein, comprising a therapeutically effective amount of an active pharmaceutical ingredient, such as a keratolytic agent. In some embodiments, a pharmaceutical composition provided herein comprises a keratolytic agent. In specific embodiments, the keratolytic agent is selected from the group consisting of benzoyl peroxide, coal tar, dithranol, salicylic acid, selenium disulfide, alpha-hydroxy acid, urea, lactic acid, boric acid, retinoic acid, sodium thioglycolate, allantoin, zinc pyrithione, zinc L-pyrrolidone carboxylate, seleocysteine, selenomethionine, captopril, zofenopril, tiopronin, penicillamine, L-cysteine, N-acetyl cysteine (NAC), gluthatione, dithiothreitol, thiorphan, cysteamine, bucillamine, dimercaprol, 1,1-ethanedithiol, dimercaptosuccinic acid, furan-2-ylmethanethiol, omapatrilat, ovothiol A, rentiapril, thiosalicylic acid, tixocortol, mycothiol, coenzyme A, coenzyme B, disulfiram, psammaplin A, dixanthogen, pantethine, fursultiamine, octotiamine, sulbutiamine, prosultiamine, thiram, lipoic acid, lenthionine, ajoene, allicin, gemopatrilat, thioethanol, thiophospholipid, thiocholesterol, 12-mercaptododecanoic acid, 23-(9-mercaptononyl)-3,6,9,12,15,18,21-heptaoxatricosanoic acid, and sulfanegen. In some embodiments, the keratolytic agent is selected from the group consisting of benzoyl peroxide, coal tar, dithranol, salicylic acid, selenium disulfide, N-acetyl cysteine (NAC), bucillamine, captopril, alpha-hydroxy acid, urea, lactic acid, sodium thioglycolate, zinc pyrithione, and zinc L-pyrrolidone carboxylate.

In some embodiments, a pharmaceutical composition provided herein comprises selenium disulfide (SeS2) (e.g., as an active pharmaceutical ingredient).

In some embodiments, a system, dispenser, or (e.g., semi-solid) pharmaceutical composition provided herein is used for treating a disease or disorder in or around an eye. In some embodiments, a system, dispenser, or (e.g., semi-solid) pharmaceutical composition provided herein is used for treating meibomian gland dysfunction (MGD), blepharitis, seborrheic blepharitis, Demodex infestation, dry eye disease (DED), hyperkeratosis, dermatitis, keratitis, contact lens discomfort, lid wiper epitheliopathy (LWE), Keratoconjunctivitis Sicca, Sjogren's Syndrome, or ocular rosacea. In specific embodiments, a system, dispenser, or (e.g., semi-solid) pharmaceutical composition provided herein is used for treating DED. In specific embodiments, a system, dispenser, or (e.g., semi-solid) pharmaceutical composition provided herein is used for treating MGD.

Provided in certain embodiments herein is a system for dispensing a semi-solid pharmaceutical composition, the system comprising the semi-solid pharmaceutical composition comprising a therapeutically effective concentration of selenium disulfide (SeS2); and a dispenser configured to dispense precise volume of the pharmaceutical composition, the volume being sufficient to treat a disease or disorder in or around the eye of an individual when the volume is administered in or around the eye of an individual, wherein the dispenser comprises a barrel chamber having an outlet and a plunger configured to dispense the pharmaceutical composition from the chamber through the outlet (e.g., as described in US 2020/0147323, US 2020/0147322, US 2022/0387731, US 2022/0134021, WO 2020/102444, and WO2020/102446, each of which is incorporated herein by such reference for such disclosure).

Provided in certain embodiments herein is a method of treating a disease or disorder in or around an eye of an individual, the method comprising dispensing a (e.g., precise) volume of a (e.g., semi-solid) pharmaceutical composition from a system provided herein and administering the dispensed (e.g., semi-solid) pharmaceutical composition in or around the eye of the individual.

Provided in certain embodiments herein is a method of treating a disease or disorder in or around an eye of an individual, the method comprising dispensing a (e.g., precise) volume of a (e.g., semi-solid) pharmaceutical composition from a system provided herein and administering the dispensed (e.g., semi-solid) pharmaceutical composition in or around the eye of the individual for up to Nₘ times (e.g., wherein Nₘ is any number provided herein). In specific embodiments, the (e.g., semi-solid) pharmaceutical composition is dispensed and administered at least 2 times. In specific embodiments, the (e.g., semi-solid) pharmaceutical composition is dispensed and administered at least 3 times. In specific embodiments, the (e.g., semi-solid) pharmaceutical composition is dispensed and administered at least 5 times. In specific embodiments, the (e.g., semi-solid) pharmaceutical composition is dispensed and administered at least 7 times. In specific embodiments, the (e.g., semi-solid) pharmaceutical composition is dispensed and administered at least 10 times. In specific embodiments, the (e.g., semi-solid) pharmaceutical composition is dispensed and administered at least 14 times. In specific embodiments, the (e.g., semi-solid) pharmaceutical composition is dispensed and administered at least 28 times. In specific embodiments, the (e.g., semi-solid) pharmaceutical composition is dispensed and administered at least 30 times.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1—Therapeutic Benefit Achieved Using Precise Dosing

Surfactant- and preservative-free ointments comprising selenium disulfide (SeS₂) (0.5 wt. %, 1 wt. %) in an oleaginous (petrolatum) base were provided. Compositions provided are provided in an amount of about 5 μL and then administered to a surface around the eye (the eyelid margin) i. Administration of the composition was provided either twice weekly or once daily. No clinical or microscopic findings were observed for any tested concentration or dosing regimen.

Such compositions are administered to the inner surface of an eyelid identified as being pre-symptomatic or symptomatic for MGD. Patients used a device to expel an exact 5 μL volume of SeS₂ ointment onto an applicator or their finger. Patients applied the ointment either twice weekly or once daily for 3 months. No serious adverse event related to treatment was observed. Most patients exhibited a substantial improvement in total Ocular Surface Disease Index (OSDI), Standard Patient Evaluation and Eye Dryness (SPEED), quality of meibum (MGS), number of open meibomian glands (MGYLS) by month 3 (data not shown).

What is claimed is:

1. A system for dispensing a semi-solid pharmaceutical composition, the system comprising:
a) the semi-solid pharmaceutical composition; and
b) a dispenser comprising (i) a chamber configured to contain a first volume (V₁) of the semi-solid pharmaceutical composition, (ii) an outlet in fluid communication with the chamber, and (iii) a device (e.g., a plunger) configured to expel a portion of the semi-solid pharmaceutical composition from the chamber and dispense a precise volume of the semi-solid pharmaceutical composition from the outlet,
wherein the portion of the semi-solid pharmaceutical composition expelled from the chamber has a second volume (V₂), the device is configured to expel a maximum number (Nₘ) of second volume portions of the semi-solid pharmaceutical composition from the chamber, and V₁>V₂×Nₘ.

2. The system of claim 1, wherein Nₘ is about 120 or less.

3. The system of claim 1, wherein Nₘ is about 30 or less.

4. The system of claim 1, wherein V₁ is at least 2 times V₂×Nₘ.

5. The system of any claim 1, wherein V₁ is at least 10 times V₂×Nₘ.

6. The system of claim 1, wherein V₂ is about 25 μL or less.

7. The system of claim 1, wherein V₂ is about 5 μL.

8. The system of claim 1, wherein the semi-solid pharmaceutical composition is an ointment, a gel, a cream, or a paste.

9. The system of claim 1, wherein the semi-solid pharmaceutical composition comprises a keratolytic agent.

10. The system of claim 1, wherein the semi-solid pharmaceutical composition comprises selenium disulfide (SeS₂) in an amount of about 0.1 wt. % to about 2 wt. %.

11. The system of claim 1, wherein the semi-solid pharmaceutical composition comprises selenium disulfide (SeS₂) in an amount of about 1 wt. %.

12. The system of claim 1, wherein the semi-solid pharmaceutical composition comprises selenium disulfide (SeS₂) in an amount of about 0.5 wt. %.

13. The system of claim 1, wherein the semi-solid pharmaceutical composition comprises selenium disulfide (SeS₂) in an amount, wherein V₂ of the semi-solid pharmaceutical composition comprises a therapeutically effective amount of the selenium disulfide (SeS₂).

14. A method of treating meibomian gland dysfunction (MGD) or dry eye disease (DED), the method comprising dispensing a precise volume of the semi-solid pharmaceutical composition from the system of claim 1 and administering the dispensed semi-solid pharmaceutical composition in or around an eye of the individual.

15. The method of claim 14, wherein the method comprises dispensing the semi-solid pharmaceutical composition for up to Nₘ times.

16. The method of claim 14, wherein the semi-solid pharmaceutical composition is an ointment.

17. The method of claim 14, wherein the semi-solid pharmaceutical composition comprises selenium disulfide in an amount of about 0.5 wt. % to about 1 wt. %.

18. The method of claim 17, wherein the semi-solid pharmaceutical composition comprises selenium disulfide in an amount of about 0.5 wt. %.

19. The method of claim 17, wherein the semi-solid pharmaceutical composition comprises selenium disulfide in an amount of about 1 wt. %.

20. A method for dispensing a semi-solid pharmaceutical composition, the method comprising:
a) providing a system comprising the semi-solid pharmaceutical composition and a dispenser, and
b) dispensing a precise volume of the semi-solid pharmaceutical composition from the dispenser;
wherein the dispenser comprises (i) a chamber configured to contain a first volume (V₁) of the semi-solid pharmaceutical composition, and (ii) an outlet in fluid communication with the chamber,
wherein the dispenser is configured to expel a portion of the semi-solid pharmaceutical composition from the chamber and dispense the precise volume of the semi-solid pharmaceutical composition from the outlet;
wherein the precise volume has a second volume (V₂);
wherein the dispenser is configured to expel a maximum number (Nₘ) of second volume portions of the semi-solid pharmaceutical composition from the chamber; and
wherein V₁>V₂×Nₘ.
